(19) United States
(12) Patent Application Publication
(10) Pub. No.: US 2022/0202631 A1
(43) Pub. Date: Jun. 30, 2022

(54) Pantiless Liner

(71) Applicant: Tara Monique Harris, Woodland Hills, CA (US)

(72) Inventor: Tara Monique Harris, Woodland Hills, CA (US)

(21) Appl. No.: 17/239,156

(22) Filed: Apr. 23, 2021

Related U.S. Application Data
(60) Provisional application No. 63/199,479, filed on Dec. 31, 2020.

(51) Int. Cl. A61F 13/56; A61F 13/472

(52) U.S. Cl. CPC A61F 13/5611; A61F 2013/8497; A61F 13/5616; A61F 13/472

(57) ABSTRACT

The invention relates to panty liners, and more particularly to a Pantiless Liner that has adhesive on the upper side. This Pantiless Liner helps to keep women dry, gives protection while maintaining a sleek look in your clothing. (1) Designs will be made with silicone or self-adhesive strips that is sensitive to the skin and will stick to the women's inner thigh. (2) Aims to produce liners that will give women the protection and comfort needed when wearing skirts, skinny jeans, leggings, and other clothing that won't show an uneven panty line when wearing pants. (3) Without having a fear of vaginal discharge or secretion in your clothing. (4) Also caters for post-intercourse discharge and slight urinary leakage due to a weak bladder. (5) Due to the silicone or adhesive strips on the upside of the product and will securely stick to the inner thigh of the body, the Pantiless Liner is an innovative product not currently on the market, it's super beneficial when young girls and women who are athletic or just lounging who has no desire to wear panties but needs the comfort and protection of vaginal because of natural discharge, secretion or moisture of the vaginal.

### BACKGROUND OF THE INVENTION

### 1. Technical Field

The present invention relates to an article of clothing. More specifically, the present invention relates to methods and apparatus of a Pantiless Liner that is used without the use of underwear/panties. The Pantiless Liner has silicone or adhesive that is sensitive for the skin on the upside of the product as it is illustrated the attached drawings. The silicone or adhesive that is sensitive to the skin will attach to the inner thigh of the woman. The Pantiless Liner is designed for young girls and women to be able to wear their clothing without wearing underwear and still have the comfort of moisture absorption with the cotton middle, protection of the vaginal from tight clothing and will also trap the aroma of the vaginal. The Pantiless Liner having silicone self-adhesive strips that will stick to the women's inner thigh which targets to produce liners that will give women the protection, comfort and moisture absorption needed when wearing skirts, dresses, skinny jeans, leggings, and other clothing that won't show an uneven panty line when wearing pants and also help women who wear tight clothing with the discomfort of the thick inseam on most jeans and pants.

### 2. Background Art

Other panty liners currently on the market only work with wearing panties or thongs. Vaginal discharge is a very common and completely natural phenomenon. Some women use panty liners to protect their underwear from vaginal discharge and maintain a dry, fresh, and clean feeling throughout the day.

A panty liner is a thin, absorbent pad worn in the gusset of underwear to absorb vaginal discharge or light menstrual flow. Panty liners are basically thin versions of pads used to prevent stains and keep underwear clean. Panty liners come in a variety of shapes, portability options, and sizes, from small, compact panty liners to large, protective panty liners created for heavy vaginal discharge and light period flow. Some styles of panty liners are designed to fit inside thongs. The purpose of panty liners is to absorb everyday vaginal discharge, unexpected light period flow, light spotting, staining at the beginning and ends of periods, and post-intercourse discharge. Tampons, and menstrual cups can be used with panty liners for extra protection. Some people find panty liners to be more flexible and comfortable than pads.

Vaginal discharge is normal and happens for a variety of reasons throughout the menstrual cycle (keeping the vagina lubricated, ovulation, and arousal, to name a few). Wearing a panty liner can be helpful for keeping underwear dry and free from stains. Having panty liners on hand may be useful during puberty, as spotting and unanticipated periods can be frequent.

Panty liners can be worn before or after periods, but they are not meant for heavy flows. Panty liners can rub against the labia, possibly triggering redness, irritation, and itching. Scented panty liners might contain chemicals that can irritate the sensitive tissues around the vagina. Both synthetic-fiber underwear and regular use of panty liners with an impermeable layer block air circulation to the reproductive organs and don't allow sweat to evaporate so therefore there is a need in the art for an article of Pantiless Liner which will not only work with wearing panties or thongs, it also gives you the freedom to stay clean and comfort without wearing panties or thongs.

### DISCLOSURE OF THE INVENTION

Briefly, and in general terms, the present invention provides a new and improved article of clothing comprising Pantiless Liner having a silicone strip, adhesive border tape to cover the vaginal area or has wings that will stick to the inner thigh that has been illustrated in the first provisional patent application. This panty liner requires no panties to use. This Pantiless Liner will stay in place wearing a dress, pants, and shorts and just lounging around the house. It comprise of a method including one or more of the following components, elements, or process steps.

This liner has adhesiveness that securely sticks to the inner thigh around the outside of the vaginal area. There will be silicone or adhesive tape that is sensitive to the skin that will secure the front of the liner as well as the back. The silicone or adhesive tape will stick all day to the inner thigh around the vaginal wall without becoming unglued with sweat, moisture or any type of wetness. The silicone strip or adhesive tape may also reattach and hold if undone to use rest room if the skin is dry.

Pantiless Liner will be made shorter in length that most liners on the market; however, this innovative Pantiless Liner will have adhesive or silicone that will stick to the women inner thigh requiring no underwear for usage. I have designed a regular, oval shape and a few fun designs of the Pantiless Liner as illustrated in the drawings. In some embodiments, the present invention may comprise of Pantiless Pads or Pantiless Sanitary Napkins that women may wear while on their menstrual cycle using the same innovative concept of the silicone or adhesive being around the border of the product for secureness that will stick to the inner thigh of the body. The present invention may comprise a panty-liner further comprising the right adhesive, shape and design that will safely and securely protect the women while they are wearing clothing without panties.

### BRIEF DESCRIPTION OF THE DRAWINGS

The following description illustrates certain embodiments of the design along with the drawings by the way of example only. The reference numbers presented herein describe the same entities of the design or the same type of the entities in the given design on all the drawings.

Referring to FIGS. 1&2, incorporated herein by reference in their entirety, a Pantiless Liner that requires no panties for use in accordance with the present invention are shown.

FIG. 1 is a front elevation of the shorten oval shape Pantiless Liner view of an article of clothing comprising Pantiless Liner having a silicone or adhesive strips around the border that will stick to the women's inner thigh. Whereas the length gathers 10 cm of the fabric with a width of 8 cm and 5 cm each from the front with a peeling sticker to stick around thighs properly to cover the vaginal to reduce discharge or secretion in your clothing while maintain comfort and protection of the vaginal. It contains a good quality fabric inside.

FIG. 2 is a front elevation of the shorten regular shape Pantiless Liner view of covering 25 cm of the length with a 7 cm width which will securely stick to the inner thigh of the body. Both drawings FIG. 1 and FIG. 2 both illustrate silicone or adhesive around the boarder of the product for secureness when using the product. FIG. 1 and FIG. 2 are both designed to be shorter than the average panty liner on the market. FIG. 1 and FIG. 2 both will provide comfort, protection, absorb moisture, trap aroma while giving relief and comfort to women with mild to moderate urine leakage due to a weak bladder.

FIG. 3 shows a sample design of a Butterfly pattern with the same quality as per client feasibility. It demonstrates the view having peel and stick paper to attach properly around inner thigh.

FIG. 4 this pantiless liner has multiple designs where design flower is one of the most comfortable design having the silicone or adhesive petals fit and stick to the inner thigh of the body.

FIG. 5 6 inches length wise lips design is super comfy when concerned about protection or comfort, this shape also has silicone or adhesive that will attach to the inner thigh of the body while running around with active daily duties.

FIG. 6 is a leaf design which depicts the front and back view of the of the liner having removable self-adhesive strips with the quilted protective center which will comfort and protect the vaginal.

While all designs have different shapes, the concept is the same; all Pantiless Liners will have cotton woven or quilted center with a border of silicone or adhesive that is sensitive to the skin that will attach to body and can be reapplied to the skin throughout the day. The innovative Pantiless Liner has adhesive on the upside of the product for secureness to the inner thigh. It can be reapplied to dry skin. The Pantiless Liner will provide young girls and women globally the same protection, comfort, absorbs moisture, help with secretion and discharged, traps the aroma of the vaginal all while not wearing underwear or panties.

### DETAILED DESCRIPTION OF THE INVENTION

Briefly, and in general terms, the present invention provides a new and improved article of clothing comprising Pantiless Liner having an silicone or adhesive tape around the border for secureness to cover the vaginal area or could have wings that will stick to the inner thigh. The following detailed description is of the best currently contemplated modes of carrying out exemplary embodiments of the invention. The description is not to be taken in a limiting sense, but is made merely for the purpose of illustrating the general principles of the present invention.

Broadly, an embodiment of the present invention provides an adhesive panty liner with wings. In one aspect the present invention may provide a panty liner that has an adhesive tape to cover the vaginal area and has wings that will stick to the inner thigh. This panty liner requires no panties to use. The present invention provides a Pantiless Liner which allows women and young ladies all over the world to feel secure with normal discharge, secretion and moisture of the vagina that most women have wear their clothing without panties.

Other panty liners currently on the market only work with wearing panties or thongs. Pantiless Liner will allow women to be pantiless free with leggings, workout wear and the like and still have the comfort of moisture absorption from the Pantiless Liner brand. The Pantiless Liner will also help women who wear tight clothing with the discomfort of the thick inseam on most jeans and pants. With the present invention, women and young ladies all over the world will feel secure and still maintain a sense of dryness with the Pantiless Liner.

The present invention differs from and distinguishes over what currently exists. Pantiless Liner is not currently on the market. Pantiless Liner will allow the Pantiless Liner to have silicone strips or adhesive tape that is sensitive to the skin on the upside that will stick to the inner vaginal wall for secure feeling. This invention is an unprecedented improvement over what currently exists. All panty liners on the market currently require wearing panties. Most women and young ladies currently do not wear panties on a daily basis. Most women only wear panties to absorb the discharge, moisture and secretion through the day.

Also, “No Panties Needed” and Pantiless Liner brand can also produce maxi pads that can be worn without panties using the same concept of the “No Panties Needed” and Pantiless Liner brand panty liners. They will be called “No Panties Needed” or Pantiless Pads, Pantiless Sanitary Napkins brand for maxi pads.

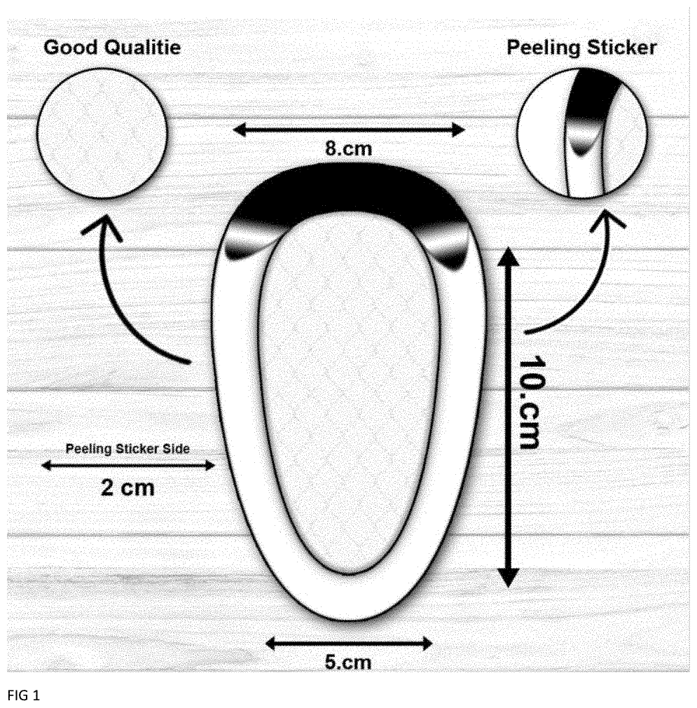

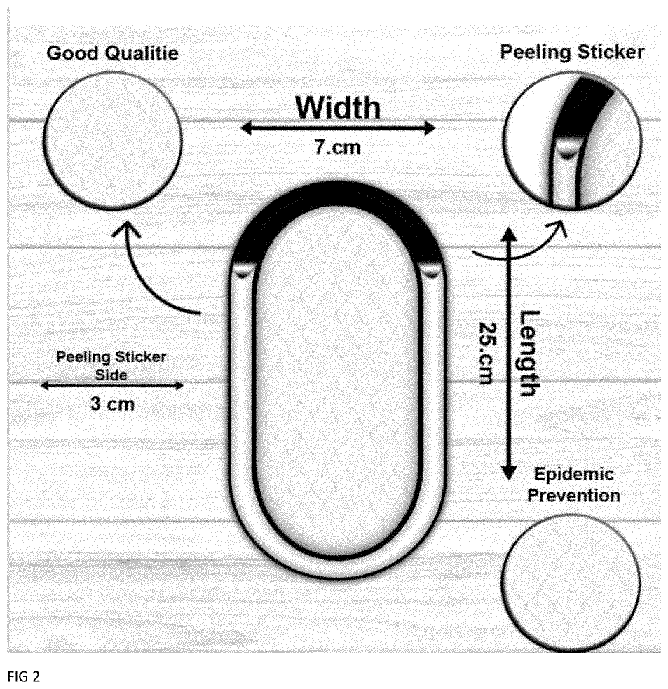

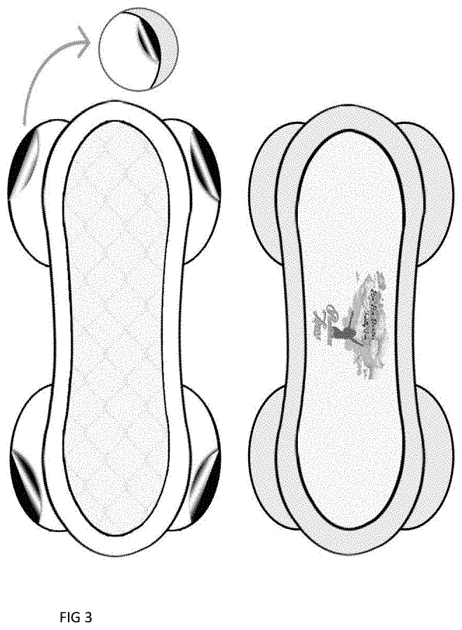

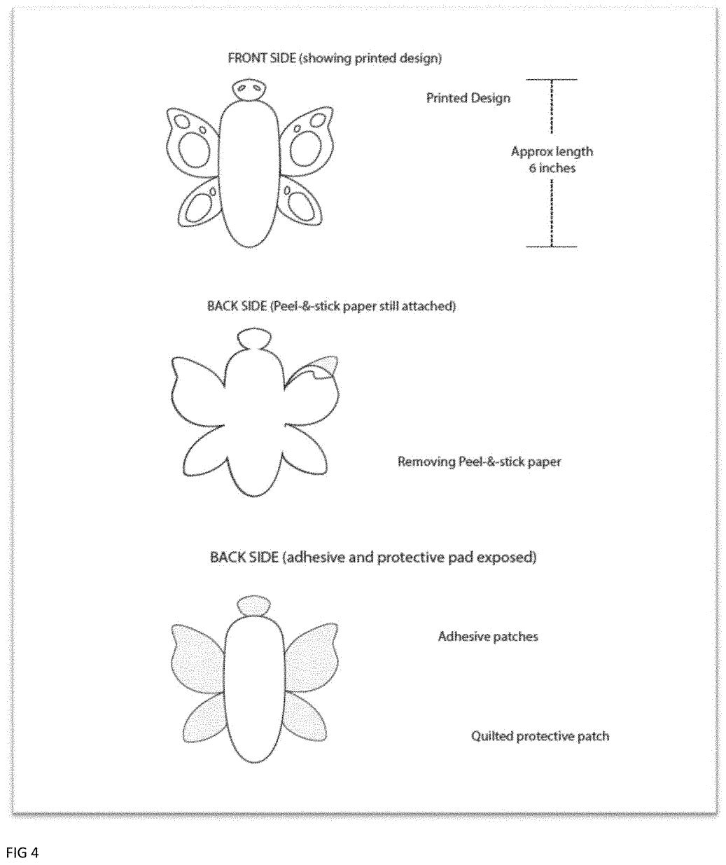

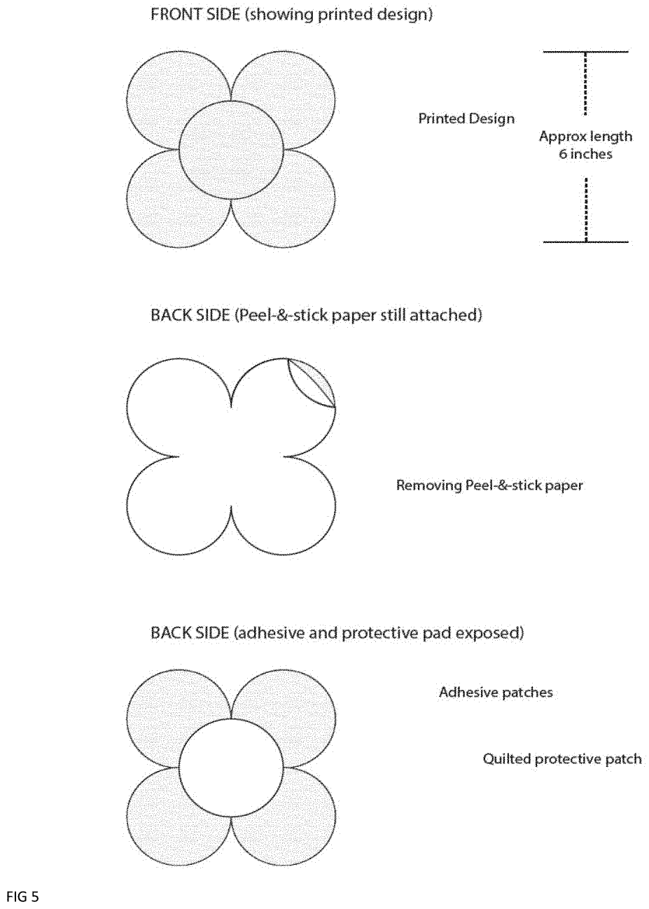

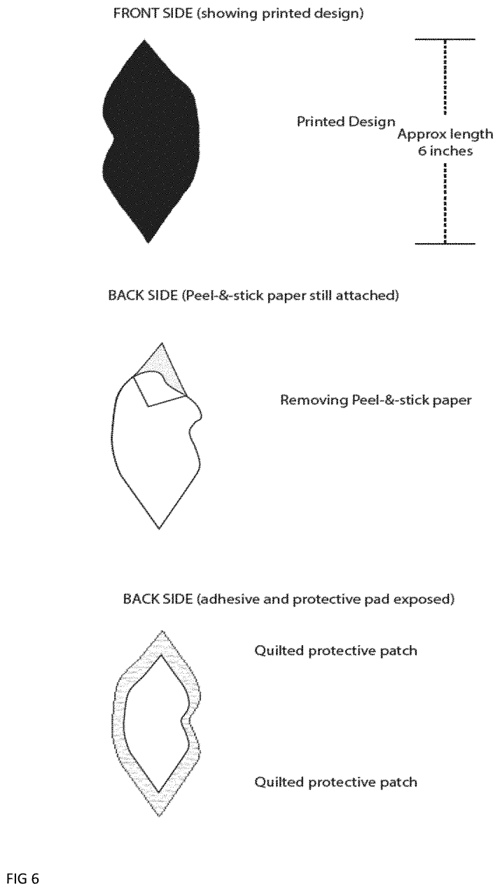

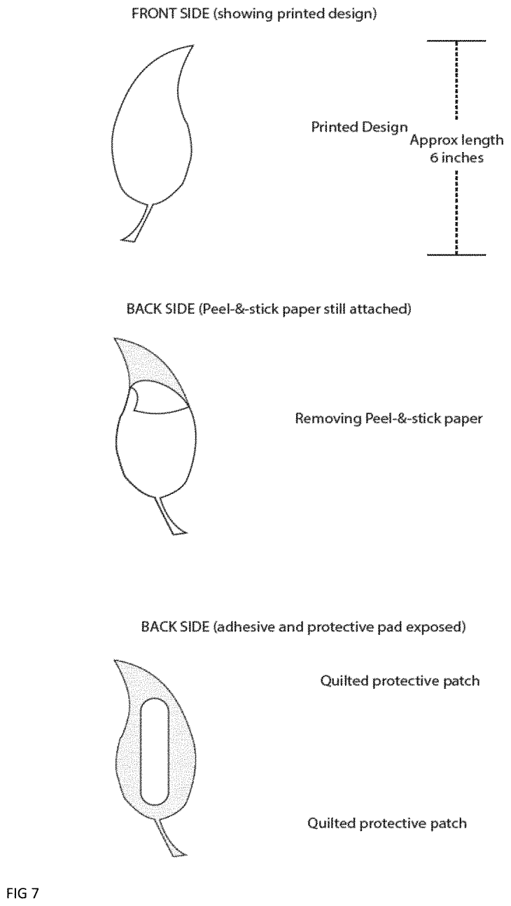

Referring to FIGS. 1&2, incorporated herein by reference in their entirety, adhesive panty liner devices in accordance with the present invention are shown.

In an exemplary embodiment the present invention may include one or more of the following components or elements. “No Panties Needed” Pantiless Liner invention and brand will first start with Pantiless Liner panty liners that are constructed and arranged to include silicone strips or adhesive tape which will be on an upper side that will be capable of adhering to stick to inner thigh around the vaginal area. This is where Pantiless Liner are needed to stay dry and feel comfortable for all women and young ladies who wants to feel free. The vaginal area will be covered with the Pantiless liner that has adhesive tape that will stick to the inner thigh around the vaginal that will endure sweat, moisture, and wetness while removing safely without pain or irritation.

Pantiless Liner will include any suitable adhesive which is capable of meeting requirements of the right adhesive tape that will stay secure while there may be sweat, moisture, wetness while maintaining a safe and less painful way if removing. Pantiless Liner will also need to test whether the adhesive is reusable if removed to urinate.

The structure may be an improvement over existing panty liners already on the market. To make the-Pantiless Liner brand stand out and be different is, self-adhesive taping or strips that will securely stick to the inner thigh around the vaginal area. Pantiless Liner brand will be made in a regular shape of panty liners and thong design or similar to.

In some embodiments, the present invention may comprise pads that women may wear while on their menstrual cycle. The present invention may comprise a panty liner further comprising the right adhesive, shape and design that will safely and securely protect the women while they are wearing clothing without panties. In an exemplary embodiment the adhesive may be capable of securely remaining in place if removed for urination, and may be reliable for use again.

Options may include small, medium or large size panty liners. The Pantiless liner may be of any suitable length. In one embodiment, to securely stay in place, the Pantiless Liner may require 2 sets of adhesive applied to both sides in the front, middle and toward the back near the rectum.

For clarity, only those aspects of the system germane to the invention are described, and product details well known in the art are omitted. In addition, many embodiments of the present invention have application to a wide range of industries. To the extent the present application discloses a system; the method implemented by that system is within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the present invention.

1. Women clothing, comprising:
a Pantiless Liner having silicone or adhesive tape to cover the vaginal area and has wings that will stick to the inner thigh.
Pantiless Liner requires no panties to use comparative to other products available in the market.
The Pantiless Liner has adhesiveness around the border or wings that securely sticks to the inner thigh around the outside of the vaginal area.

2. The garments of claim 1, wherein the adjustment member comprises a peeling sticker.

3. The garment of claim 1, wherein the adjustment member comprises wings that will stick inside to absorb moisture.

4. The garment of claim 1, wherein the adhesive tapes is manufactured in a material also being elastic.

5. Women's garment, comprising:
adhesive tape will stick all day to the inner thigh around the vaginal wall without becoming unglued with sweat, moisture or any type of wetness.
The adhesive tape may also reattach and holds if undone to use rest room. liner is to be made in a regular design and size of the thong or similar to as shown in drawings.
a panty-liner further comprising the right adhesive, shape and design.

6. The garment of claim 5, wherein the adjustment member comprises sufficient calculated length* width as shown in FIG. 1 to get the protection done.

7. The garment of claim 1, wherein the adhesive is reusable if removed to urinate.

8. The garment of claim 5, wherein the invention is an unprecedented improvement over what currently exists.
